(19) United States
(12) Patent Application Publication
(10) Pub. No.: US 2025/0303014 A1
(43) Pub. Date: Oct. 2, 2025

(54) ELECTRICALLY HEATED DIRI WITH CONTROLLED VOLATILIZATION

(71) Applicant: Micheon Eubanks, San Leandro, CA (US)

(72) Inventor: Micheon Eubanks, San Leandro, CA (US)

(21) Appl. No.: 19/238,575

(22) Filed: Jun. 16, 2025

Related U.S. Application Data
(63) Continuation-in-part of application No. 17/687,047, filed on Mar. 4, 2022.

(51) Int. Cl. A61L 9/03

(52) U.S. Cl. CPC A61L 9/03; A61L 2209/135

(57) ABSTRACT

A diri comprises a thermally conductive rivet body having top and bottom openings defining a scent passage therethrough. A heating element comprising a conductive wire coiled around the rivet body includes positive and negative terminals for electrical connection. A scent substance comprising volatile organic compounds is disposed within the rivet body between the openings. Upon application of electrical current to the heating element, heat is generated sufficient to volatilize the scent substance and release scent vapor through the openings. The apparatus includes a temperature regulation mechanism maintaining heating element temperature within predetermined parameters to ensure consistent volatilization rates. The rivet body exhibits thermal conductivity of at least 50 W/m·K, facilitating efficient heat transfer from the heating element to the scent substance. The apparatus achieves controlled scent release rates and vapor output efficiency of at least 70% conversion within 30 seconds of electrical activation.

### BACKGROUND OF THE INVENTION

### Field of Invention

The various aspects discussed herein relate to diri and methods for controlled release of aromatic substances.

### Description of Related Art

Conventional Existing scent delivery systems suffer from several limitations that impact their effectiveness and user experience. Conventional scent dispensing devices often lack precise control over volatilization rates, resulting in inconsistent scent release that may be either too weak to be perceptible or overwhelmingly strong. Additionally, many existing solutions fail to provide efficient heat transfer mechanisms, leading to incomplete volatilization of scent substances and wasteful consumption of aromatic materials. Current systems also frequently exhibit poor temperature regulation, which can cause thermal degradation of delicate scent compounds and reduce the quality of the released fragrance. Furthermore, conventional diri typically lack the compact form factor and reliable electrical connections necessary for integration into various applications and environments.

Accordingly, there is a need in the art for an improved diri that provides controlled, efficient volatilization of scent substances with precise temperature regulation and reliable electrical connectivity in a compact configuration.

### BRIEF SUMMARY OF THE INVENTION

This summary is provided to introduce a selection of concepts, in a simplified format, that are further described in the detailed description of the invention. This summary is neither intended to identify key or essential inventive concepts of the invention nor is it intended for determining the scope of the invention.

In one aspect, the present invention provides a diri that addresses the limitations of existing scent dispensing systems through a novel rivet-based design with integrated heating functionality. The apparatus comprises a rivet body having a top opening and a bottom opening that define a scent passage therethrough, a heating element comprising a conductive wire coiled around at least a portion of the rivet body with positive and negative terminals for electrical connection, and a scent substance disposed within the rivet body between the openings. When electrical current is applied to the heating element, heat is generated sufficient to volatilize the scent substance and release scent vapor through at least one of the openings.

The present invention solves the problems associated with conventional scent delivery systems by providing precise control over volatilization through the strategically positioned heating element that enables uniform heat distribution along the scent passage. Advantageously, the rivet body may be fabricated from thermally conductive metals such as aluminum, copper, brass, or steel alloys, facilitating efficient heat transfer from the heating element to the scent substance while maintaining a compact form factor. The conductive wire may comprise nichrome wire with controlled resistance characteristics operating at optimized temperatures to provide controlled volatilization without thermal degradation of delicate scent compounds.

The present invention improves upon the prior art by incorporating temperature regulation mechanisms that maintain consistent volatilization rates and prevent overheating, while the precisely dimensioned openings create controlled vapor flow paths that regulate scent release rates. The apparatus achieves superior conversion efficiency of scent substance to released vapor within rapid timeframes, addressing the wasteful consumption issues of existing solutions. Additionally, the corrosion-resistant electrical contacts provide reliable electrical connectivity and resistance to thermal cycling degradation, ensuring long-term operational stability.

In another aspect, the present invention provides a method for controlled scent release that enables precise modulation of electrical current to achieve desired scent output levels, providing users with unprecedented control over their aromatic environment. The invention has broad applicability across various industries including automotive, residential, commercial, and personal care applications, offering enhanced user experience through consistent, efficient, and controllable scent delivery in a compact, reliable apparatus.

Additional features and advantages of the invention will be set forth in the description which follows. These and other features of the present invention will become more fully apparent from the following description, or may be learned by the practice of the invention as set forth hereinafter.

### BRIEF DESCRIPTION OF THE DRAWINGS

The various exemplary embodiments of the present invention, which will become more apparent as the description proceeds, are described in the following detailed description in conjunction with the accompanying drawings, in which:

FIG. 1 is a perspective view illustrating a diri for controlled volatilization and release of aromatic substances.

FIG. 2 is a flowchart illustrating the system information and user flow diagram for the diri 100 of FIG. 1.

### DETAILED DESCRIPTION

In the following detailed description of the preferred embodiments, reference is made to the accompanying drawings, which form a part hereof and show, by way of illustration, specific embodiments in which the invention may be practiced. It is to be understood that other embodiments may be used and structural or logical changes may be made without departing from the scope of the present invention. The following detailed description, therefore, is not to be taken in a limiting sense, and the scope of the present invention is defined by the appended claims.

The following description is provided as an enabling teaching of the present systems, and/or methods in its best, currently known aspect. To this end, those skilled in the relevant art will recognize and appreciate that many changes can be made to the various aspects of the present systems described herein, while still obtaining the beneficial results of the present disclosure. It will also be apparent that some of the desired benefits of the present disclosure can be obtained by selecting some of the features of the present disclosure without utilizing other features.

Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

The terms “a” and “an” and “the” and similar references used in the context of describing a particular embodiment of the present invention (especially in the context of certain claims) are construed to cover both the singular and the plural. The recitation of ranges of values herein is merely intended to serve as a shorthand method of referring individually to each separate value falling within the range. Unless otherwise indicated herein, each individual value is incorporated into the specification as if it were individually recited herein.

All systems described herein can be performed in any suitable order unless otherwise indicated herein or otherwise clearly contradicted by context. The use of any and all examples, or exemplary language (for example, “such as”) provided with respect to certain embodiments herein is intended merely to better illuminate the application and does not pose a limitation on the scope of the application otherwise claimed. No language in the specification should be construed as indicating any non-claimed element essential to the practice of the application. Thus, for example, reference to “an element” can include two or more such elements unless the context indicates otherwise.

As used herein, the terms “optional” or “optionally” mean that the subsequently described event or circumstance can or cannot occur, and that the description includes instances where said event or circumstance occurs and instances where it does not.

The word or as used herein means any one member of a particular list and also includes any combination of members of that list. Further, one should note that conditional language, such as, among others, “can,” “could,” “might”, or “may” unless specifically stated otherwise, or otherwise understood within the context as used, is generally intended to convey that certain aspects include, while other aspects do not include, certain features, elements and/or steps. Thus, such conditional language is not generally intended to imply that features, elements and/or steps are in any way required for one or more particular aspects or that one or more particular aspects necessarily include logic for deciding, with or without user input or prompting, whether these features, elements and/or steps are included or are to be performed in any particular aspect.

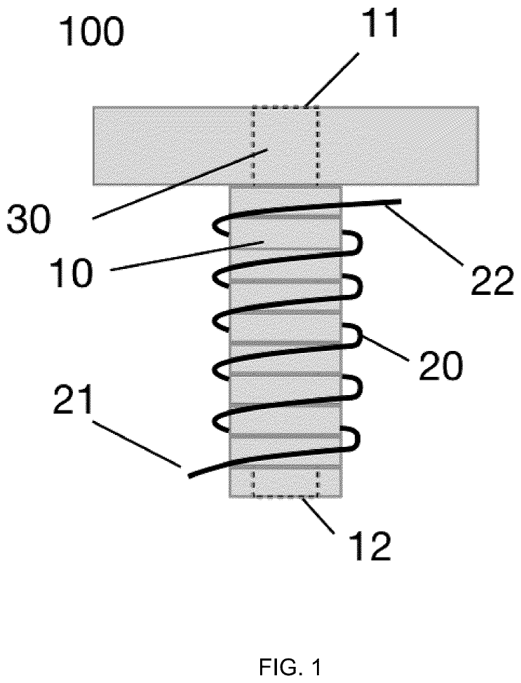

FIG. 1 illustrates FIG. 1 is a perspective view illustrating a diri 100 for controlled volatilization and release of aromatic substances.

The apparatus comprises a rivet body 10 having a generally cylindrical configuration with a top opening 11 and a bottom opening 12 defining a continuous scent passage therethrough. The rivet body 10 is fabricated from a thermally conductive metal selected from aluminum, copper, brass, or steel alloys, exhibiting a thermal conductivity of at least 50 W/m·K to facilitate efficient heat transfer throughout the apparatus structure.

Disposed around the exterior surface of the rivet body 10 is a heating element 20 comprising a conductive wire configured in a helical coil arrangement. The conductive wire 20 is formed from nichrome material having a resistance range of 0.5 to 5.0 ohms per foot and is capable of operating at temperatures between 100° C. and 300° C. The coiled configuration comprises 5 to 20 turns wrapped around the rivet body 10, with uniform spacing of 0.5 to 2.0 millimeters maintained between adjacent coils to ensure consistent heat distribution along the scent passage.

The heating element 20 terminates in a positive terminal 21 and a negative terminal 22 positioned for electrical connection. These terminals 21, 22 comprise corrosion-resistant electrical contacts fabricated from gold-plated copper, silver-plated copper, or nickel-plated brass, providing reliable electrical connectivity and resistance to thermal cycling degradation.

Contained within the internal cavity of the rivet body 10, between the top opening 11 and bottom opening 12, is a scent substance 30 comprising volatile organic compounds having boiling points between 50° C. and 250° C. The scent substance 30 is selected from essential oils, synthetic fragrances, terpenes, or aromatic esters optimized for controlled volatilization.

The top opening 11 and bottom opening 12 each feature precisely dimensioned apertures having diameters ranging from 0.5 to 5.0 millimeters, creating a controlled vapor flow path that regulates scent release rate between 0.01 and 1.0 milligrams per minute. The rivet body 10 defines an internal volume of 0.1 to 2.0 cubic centimeters, optimally sized for efficient scent substance containment and vaporization.

Integrated within the apparatus is a temperature regulation mechanism 40 configured to maintain heating element temperature within ±5° C. of a predetermined setpoint. This mechanism 40 ensures consistent volatilization rate and prevents thermal degradation of the scent substance 30.

Upon application of electrical current of 0.1 to 5.0 amperes across the positive terminal 21 and negative terminal 22, the heating element 20 generates heat output of 1 to 50 watts. This controlled heat generation raises the temperature of the rivet body 10 and subsequently the scent substance 30 to volatilization temperatures, causing the formation of scent vapor that is expelled through the top opening 11 and bottom opening 12.

The apparatus achieves scent vapor output efficiency of at least 70% conversion of scent substance 30 to released vapor within 30 seconds of electrical current application. The thermally conductive properties of the rivet body 10 facilitate rapid and uniform heat transfer from the heating element 20 to the scent substance 30, ensuring consistent and controlled aromatic release without thermal degradation of the volatile compounds.

The helical configuration of the heating element 20 maximizes surface contact with the rivet body 10 while maintaining structural integrity during thermal cycling operations, enabling sustained and reliable scent delivery performance.

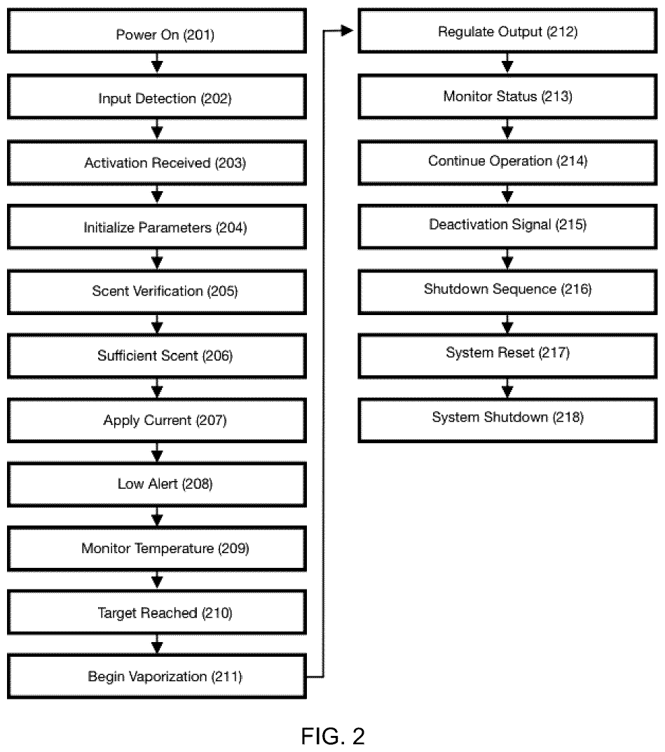

FIG. 2 is a flowchart illustrating the system information and user flow diagram for the diri 100 of FIG. 1. The flowchart contains twelve rectangular process elements, three diamond-shaped decision points, two oval start/end elements, and one parallelogram input element, all connected by unidirectional arrows depicting the sequential flow of user interactions and corresponding system responses. The diagram details the complete operational cycle from user activation through scent delivery termination, including backend information processing and control mechanisms.

The operational sequence initiates at the “System Power On” start state (201), which flows directly to the “User Input Detection” process (202). The user input detection process (202) continuously monitors for user activation signals and subsequently flows to the “Activation Command Received” decision point (203). At this decision juncture (203), if an activation signal is detected, the flow proceeds to the “Initialize System Parameters” process (204), whereas if no activation signal is present, the flow returns to the “User Input Detection” process (202) to maintain continuous monitoring capability.

The “Initialize System Parameters” process (204) configures the temperature regulation mechanism 40 settings and establishes electrical connection parameters for the positive terminal 21 and negative terminal 22, thereafter flowing to the “Scent Substance Verification” process (205). The scent substance verification process (205) detects the presence and quantity of scent substance 30 contained within the rivet body 10 and flows to the “Sufficient Scent Available” decision point (206). This decision point (206) evaluates whether the scent substance 30 quantity is adequate for operation, directing the flow to the “Apply Electrical Current” process (207) if sufficient scent is available, or alternatively to the “Display Low Scent Alert” process (208) if the scent substance 30 is insufficient.

When the “Display Low Scent Alert” process (208) is activated, it generates a user notification regarding scent substance 30 depletion and flows directly to the “System Shutdown” end state (218). Conversely, the “Apply Electrical Current” process (207) delivers electrical current ranging from 0.1 to 5.0 amperes across the positive terminal 21 and negative terminal 22, thereby activating the heating element 20 with heat output of 1 to 50 watts, and subsequently flows to the “Monitor Temperature” process (209).

The temperature monitoring process (209) continuously measures the rivet body 10 temperature via the temperature regulation mechanism 40, maintaining the heating element 20 temperature within ±5° C. of the predetermined setpoint, and flows to the “Target Temperature Reached” decision point (210). This decision point (210) determines whether optimal volatilization temperature has been achieved, directing the flow to the “Begin Scent Vaporization” process (211) if the target temperature is reached, or returning to the “Apply Electrical Current” process (207) if additional heating is required.

Upon reaching the “Begin Scent Vaporization” process (211), the system initiates controlled volatilization of the scent substance 30 within the rivet body 10, generating scent vapor release through the top opening 11 and bottom opening 12 while achieving 70% conversion efficiency within 30 seconds of activation. This process flows to the “Regulate Vapor Output” process (212), which controls the scent release rate between 0.01 and 1.0 milligrams per minute and maintains consistent vapor flow through the precisely dimensioned apertures, subsequently flowing to the “Monitor System Status” process (213)

The system status monitoring process (213) continuously evaluates heating element 20 performance, tracks scent substance 30 consumption levels, and monitors temperature regulation mechanism 40 functionality, flowing to the “Continue Operation” process (214). The continue operation process (214) maintains steady-state scent delivery operation while processing real-time system feedback and adjustments, and flows to the “User Deactivation Signal” input element (215). Upon receiving a user termination command through the deactivation signal input (215), the flow proceeds to the “Shutdown Sequence” process (216).

The shutdown sequence process (216) discontinues electrical current to the heating element 20, initiates controlled cooling of the rivet body 10, and terminates scent substance 30 volatilization, flowing to the “System Reset” process (217). The system reset process (217) returns all system parameters to standby configuration and clears operational memory and status indicators before flowing to the “System Shutdown” end state (218), thereby completing the operational cycle.

The flowchart demonstrates the complete information flow architecture wherein user inputs trigger corresponding system responses through the diri 100 components. The backend processing encompasses continuous monitoring of thermal conditions, electrical parameters, and scent substance levels to ensure optimal performance of the rivet body 10, heating element 20, and temperature regulation mechanism 40. The depicted flow integrates user interface elements with the physical apparatus components described in FIG. 1, providing a comprehensive operational framework for controlled aromatic substance delivery through systematic process management and real-time system optimization.

The embodiments described herein are given for the purpose of facilitating the understanding of the present invention and are not intended to limit the interpretation of the present invention. The respective elements and their arrangements, materials, conditions, shapes, sizes, or the like of the embodiment are not limited to the illustrated examples but may be appropriately changed. Further, the constituents described in the embodiment may be partially replaced or combined together.

What is claimed is:

1. A diri comprising:
(i) a rivet body having a top opening and a bottom opening defining a scent passage therethrough;
(ii) a heating element comprising a conductive wire coiled around at least a portion of the rivet body, the conductive wire having a positive terminal and a negative terminal for electrical connection;
(iii) a scent substance disposed within the rivet body between the top opening and the bottom opening; and
(iv) wherein application of electrical current to the heating element generates heat sufficient to volatilize the scent substance and release scent vapor through at least one of the top opening and the bottom opening.

2. The diri of claim 1, wherein the rivet body is fabricated from a thermally conductive metal selected from the group consisting of aluminum, copper, brass, and steel alloys, having a thermal conductivity of at least 50 W/m·K to facilitate efficient heat transfer from the heating element to the scent substance.

3. The diri of claim 1, wherein the conductive wire comprises nichrome wire having a resistance range of 0.5 to 5.0 ohms per foot and operating at temperatures between 100° C. and 300° C. to provide controlled volatilization of the scent substance.

4. The diri of claim 3, wherein the conductive wire is coiled in a helical configuration with 5 to 20 turns around the rivet body, maintaining uniform spacing of 0.5 to 2.0 millimeters between adjacent coils to ensure consistent heat distribution along the scent passage.

5. The diri of claim 1, wherein the scent substance comprises volatile organic compounds having boiling points between 50° C. and 250° C., selected from the group consisting of essential oils, synthetic fragrances, terpenes, and aromatic esters.

6. The diri of claim 1, wherein the heating element generates heat output of 1 to 50 watts when electrical current of 0.1 to 5.0 amperes is applied across the positive and negative terminals, sufficient to achieve volatilization temperatures without thermal degradation of the scent substance.

7. The diri of claim 1, wherein the top opening and bottom opening each have diameters ranging from 0.5 to 5.0 millimeters, creating a controlled vapor flow path that regulates scent release rate between 0.01 and 1.0 milligrams per minute.

8. The diri of claim 1, wherein the positive terminal and negative terminal comprise corrosion-resistant electrical contacts selected from gold-plated copper, silver-plated copper, or nickel-plated brass, providing reliable electrical connection and resistance to thermal cycling degradation.

9. The diri of claim 1, further comprising a temperature regulation mechanism that maintains heating element temperature within ±5° C. of a predetermined setpoint to ensure consistent volatilization rate and prevent overheating of the scent substance.

10. The diri of claim 1, wherein the rivet body has an internal volume of 0.1 to 2.0 cubic centimeters and the apparatus achieves scent vapor output efficiency of at least 70% conversion of scent substance to released vapor within 30 seconds of electrical current application.

11. A method for controlled scent release comprising:
(i) providing a rivet structure containing a scent substance and having a top hole and a bottom hole;
(ii) applying electrical current to a heating wire coiled around the rivet structure, the heating wire having positive and negative connection points;
(iii) generating heat from the electrical current sufficient to volatilize at least a portion of the scent substance within the rivet structure;
(iv) releasing scent vapor from the volatilized scent substance through the top hole and the bottom hole of the rivet structure; and
(v) controlling the scent release by modulating the electrical current applied to the heating wire.

12. The method of claim 11, wherein providing the rivet structure comprises providing a rivet fabricated from a thermally conductive metal selected from the group consisting of aluminum, copper, brass, and steel alloys, having a thermal conductivity of at least 50 W/m·K to facilitate efficient heat transfer during the generating heat step.

13. The method of claim 11, wherein the heating wire comprises nichrome wire having a resistance range of 0.5 to 5.0 ohms per foot, and wherein applying electrical current comprises operating the heating wire at temperatures between 100° C. and 300° C. to provide controlled volatilization of the scent substance.

14. The method of claim 13, wherein applying electrical current comprises energizing the heating wire coiled in a helical configuration with 5 to 20 turns around the rivet structure, maintaining uniform spacing of 0.5 to 2.0 millimeters between adjacent coils to ensure consistent heat distribution during the generating heat step.

15. The method of claim 11, wherein providing the rivet structure comprises filling the rivet structure with a scent substance comprising volatile organic compounds having boiling points between 50° C. and 250° C., selected from the group consisting of essential oils, synthetic fragrances, terpenes, and aromatic esters.

16. The method of claim 11, wherein generating heat comprises producing heat output of 1 to 50 watts when applying electrical current of 0.1 to 5.0 amperes across the positive and negative connection points, sufficient to achieve volatilization temperatures without thermal degradation of the scent substance.

17. The method of claim 11, wherein releasing scent vapor comprises directing vapor flow through the top hole and bottom hole each having diameters ranging from 0.5 to 5.0 millimeters, creating a controlled vapor flow path that regulates scent release rate between 0.01 and 1.0 milligrams per minute.

18. The method of claim 11, wherein applying electrical current comprises connecting to positive and negative connection points comprising corrosion-resistant electrical contacts selected from gold-plated copper, silver-plated copper, or nickel-plated brass, providing reliable electrical connection and resistance to thermal cycling degradation.

19. The method of claim 11, wherein controlling the scent release comprises maintaining heating wire temperature within ±5° C. of a predetermined setpoint through modulating the electrical current to ensure consistent volatilization rate and prevent overheating of the scent substance.

20. The method of claim 11, wherein providing the rivet structure comprises providing a rivet having an internal volume of 0.1 to 2.0 cubic centimeters, and wherein the method achieves scent vapor output efficiency of at least 70% conversion of scent substance to released vapor within 30 seconds of applying electrical current.
